(19) United States
(12) Patent Application Publication
(10) Pub. No.: US 2024/0358411 A1
(43) Pub. Date: Oct. 31, 2024

(54) SYSTEMS AND METHODS FOR USING A FEMORAL ARRAY CLAMP

(71) Applicants: Smith & Nephew, Inc., Memphis, TN (US); Smith & Nephew Orthopaedics AG, Zug (CH); Smith & Nephew Asia Pacific Pte. Limited, Singapore (SG)

(72) Inventor: Paul Alexander TORRIE, Marblehead, MA (US)

(73) Assignees: Smith & Nephew, Inc., Memphis, TN (US); Smith & Nephew Orthopaedics AG, Zug (CH); Smith & Nephew Asia Pacific Pte. Limited, Singapore (SG)

(21) Appl. No.: 18/687,502

(22) PCT Filed: Oct. 10, 2022
(86) PCT No.: PCT/US2022/046177

§ 371 Date: Feb. 28, 2024

Related U.S. Application Data
(60) Provisional application No. 63/276,859, filed on Nov. 8, 2021.

(51) Int. Cl. A61B 17/64; A61B 17/72; A61B 34/20

(52) U.S. Cl. CPC A61B 17/6466; A61B 17/72; A61B 34/20

(57) ABSTRACT

Systems and methods for using a femoral array clamp can include sliding a first throughbore of a first femoral array clamp on a first bone pin having a distal end disposed within a bone: inserting an array arm through a second throughbore of the first femoral array clamp; and locking a physical relationship between the first bone pin and the array arm by actuating a first fastener of the first femoral array clamp.

### CROSS-REFERENCE TO RELATED APPLICATIONS

This application claims the benefit of U.S. Provisional Application No. 63/276,859 filed 8 Nov. 2021 title “Systems and Methods for Using a Femoral Array Clamp.” The provisional application is incorporated by reference herein as if reproduced in full below.

### BACKGROUND

In computer-assisted surgical procedures, the location of various bones of a patient may be tracked by external systems. In a specific example of hip arthroscopic surgery, the location of the femur is tracked in relation to other instruments. In the related art, tracking the femur may be accomplished by tracking arrays (e.g., optical tracking arrays) that are rigidly connected to the femur by way of a bone pin through the skin. Current femur tracking equipment and techniques are limited to placement at the distal portion of the femur that is close to the knee. Although such solutions are workable, improvements in femur tracking equipment and techniques continue to be of interest.

### SUMMARY

Various examples are directed to methods and systems for using a femoral array clamp. One example of a method for using a femoral array clamp includes sliding a first throughbore of a first femoral array clamp around a first bone pin having a distal end disposed within a bone; inserting an array arm through a second throughbore of the first femoral array clamp; and locking a physical relationship between the first bone pin and the array arm by actuating a first fastener of the first femoral array clamp.

One example of a femoral array clamp includes a housing and a pin channel that extends through the housing and has a pin channel axis. A throughbore also extends through the housing. A passageway couples the pin channel and the throughbore, and the passageway has a passageway axis. A counterbore extends in the housing and has a counterbore axis, wherein the counterbore intersects the pin channel, and the counterbore axis is parallel to the passageway axis. A fastener is disposed in the counterbore. In addition, a swage assembly disposed in the throughbore, and a portion of the swage assembly protrudes through the passageway into the pin channel.

Embodiments of a system for surgical repair of a femur can include a femoral array clamp comprising a housing, and a pin channel that extends through the housing and has a pin channel axis. A throughbore can extends through the housing. A passageway can extend between the pin channel and the throughbore, and the passageway has a passageway axis. A counterbore is in the housing with a counterbore axis. The counterbore can intersect the pin channel and the counterbore axis is parallel to the passageway axis. A fastener is disposed in the counterbore. A swage assembly is in the throughbore. A portion of the swage assembly can protrude through the passageway into the pin channel. A bone pin is configured to be located in the pin channel, and an array can traverse the throughbore.

### BRIEF DESCRIPTION OF THE DRAWINGS

For a detailed description of the various embodiments, reference will now be made to the accompanying drawings.

FIGS. 1-3 are various perspective views of a clamp system in accordance with some embodiments.

FIG. 4 is a perspective view of an example of a femoral array clamp with a reference coordinate system, in accordance with at least some embodiments.

FIG. 5 is a sectional view of an embodiment of a femoral array clamp.

FIG. 6 is a sectional side view of an embodiment of a clamp system and depicts the forces between the components of the system.

FIG. 7 is a sectional view of a femoral array clamp having an array throughbore, in accordance with at least some embodiments.

FIGS. 8 and 9 are perspective views of embodiments of a clamp system showing ranges of motion for bone pins with respect to the clamp system.

FIG. 10 is a sectional top view of another example of a femoral array clamp.

FIGS. 11-14 are sectional perspective views of other examples of femoral array clamps, in accordance with some embodiments.

FIGS. 15-17 are perspective and sectional views, respectively, of embodiments of pin housings for a femoral array clamp.

FIG. 18 is a perspective view of another femoral array clamp system in accordance with at least some embodiments.

FIG. 19 is a flow chart of an embodiment of a method of using a femoral array clamp system.

FIGS. 20-23 are schematic, perspective views of sequential steps, respectively, of a method of using an embodiment of the system during operation.

### DEFINITIONS

Various terms are used to refer to particular system components. Different companies may refer to a component by different names—this document does not intend to distinguish between components that differ in name but not function. In the following discussion and in the claims, the terms “including” and “comprising” are used in an open-ended fashion, and thus should be interpreted to mean “including, but not limited to . . . ”

The term “couple” or “couples” is intended to mean either an indirect or direct connection. Thus, if a first device couples to a second device, that connection may be through a direct connection or through an indirect connection via other devices and connections.

“Throughbore” shall mean an aperture or passageway through an underlying device. However, the term “throughbore” shall not be read to imply any method of creation. Thus, a throughbore may be created in any suitable way, such as drilling, boring, laser drilling, or casting.

“Counterbore” shall mean an aperture or passageway into an underlying device. In cases in which the counter bore intersects another aperture (e.g., a throughbore), the counter bore may thus define an internal shoulder. However, the term “counterbore” shall not be read to imply any method of creation. A counterbore may be created in any suitable way, such as drilling, boring, laser drilling, or casting.

### DETAILED DESCRIPTION

The following discussion is directed to various embodiments of the invention. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

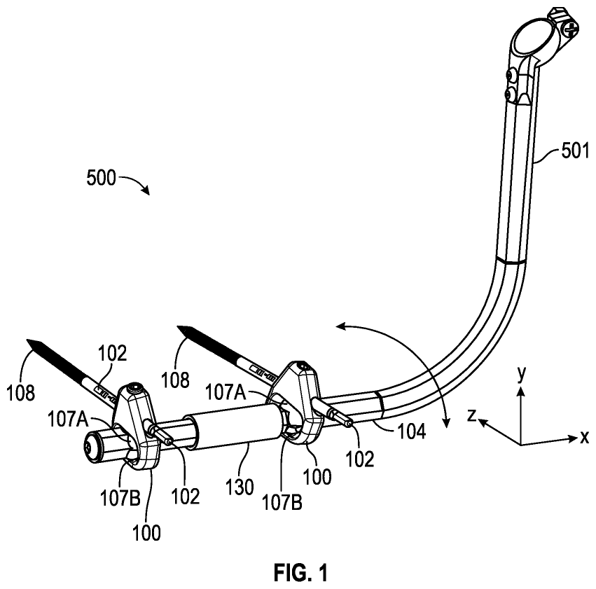

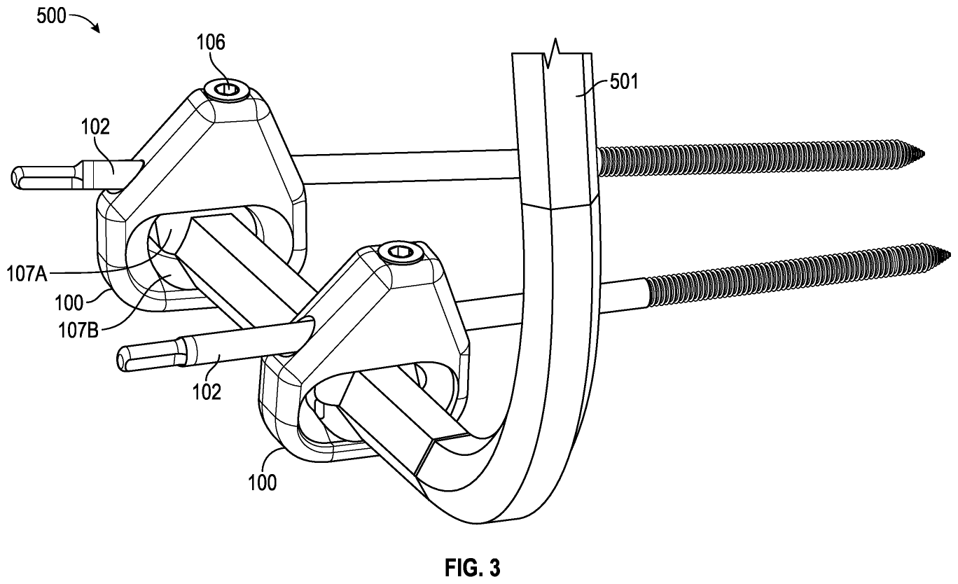

FIGS. 1-23 depict embodiments of a system, method and apparatus for assisting a surgeon during repair of a femur. For example, FIG. 1 depicts an embodiment of a system 500 that can assist a surgeon to position an external support array relative to the femur of a patient. In particular, versions of system 500 can be positioned at the more advantageous, proximal and intermediate locations on the femur, rather than only the distal portions (e.g., those closest to the knee) of the femur like conventional solutions.

Embodiments of the system 500 can include a clamp assembly having one or more clamps 100 (e.g., a femoral array clamp) that can be rigidly attached to bone pins 102 to support and lock a fiducial or tracking array for navigation with minimal impact on the many varied surgical workflows. The clamp assembly of system 500 can provide a rigid bridge between the bone pins 102 and the tracking array.

In an example, the system 500 can enable a surgeon to determine the location of bone pins 102 without having a preset configuration or location of the bone pins 102 relative to the array arm 104. In some cases a single clamp 100 may be used in the system 500. In other cases, and as shown in FIGS. 1-3, system 500 may include two (or more) clamps 100. In an instance where more than one clamp 100 is utilized, a method for using the second femoral array clamp 100 may include sliding pin channels 101 of the respective clamps 100 over respective bone pins 102 installed in the underlying bone. An array arm 104 can be inserted through respective throughbores 103 of the clamps 100. A physical relationship between the bone pins 102 and the array arm 104 can be locked by actuating respective fasteners 106 in the clamps 100.

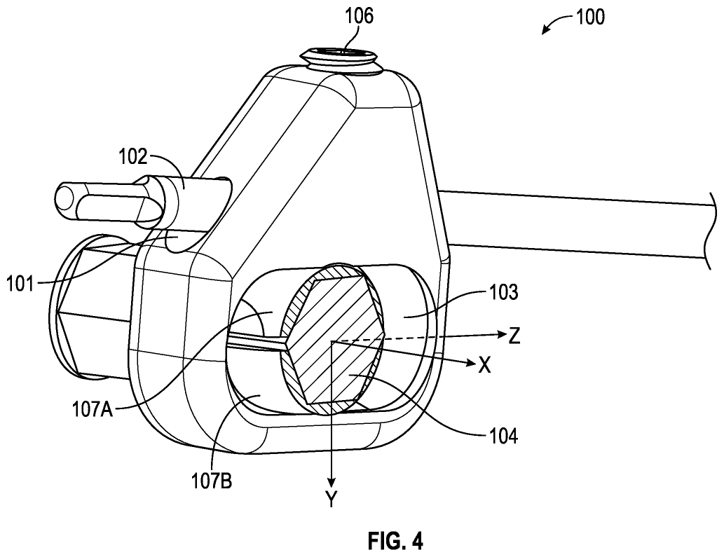

FIG. 4 includes an enlarged example of a clamp 100 that can be used in the system 500. The clamp 100 enables the surgeon to determine the location of a respective bone pin 102 without having a preset configuration or number of bone pins 102 that must be utilized. Thus, the surgeon is capable of determining, controlling, and using his or her own desired positioning for the distal ends 108 of bone pins 102 during surgery.

Some example clamps and related systems are designed and constructed such that, once the correct orientation between the clamp, the pin, and the array is achieved, that orientation can be fixed by way of the completion of one operation (e.g., tightening a single screw or inserting a single pin on each clamp).

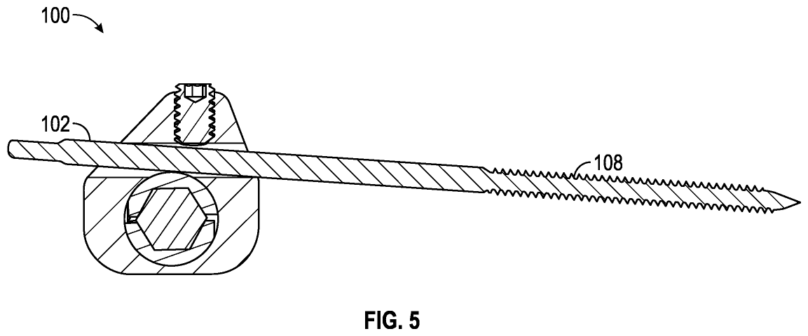

As shown in FIGS. 5-7, the clamp 100 can establish a rigid bridge between the bone pins 102 and array arm 104 for surgery. This enables the bone pins 102 to be installed independently into respective clamps 100. Since embodiments of the clamp 100 permit the bone pin 102 to be oriented in almost any trajectory near the bone location, example devices reduce the chances of harm or error when inserting bone pins 102 into the greater trochanter region of the femur.

Embodiments of the clamp 100 can include component pieces that may fit with and within the clamp 100. For example, the clamp 100 may include a housing or clamp body with channels that can be operatively coupled. In some versions, the clamp 100 may include pin channel 101 that extends through the clamp 100 in a z-axis direction (FIG. 4). The pin channel 101 may act as a throughbore of the clamp 100 to slide along a respective bone pin 102. To allow the bone pin 102 to enter the pin channel 101, the clamp 100 can be adjusted to match the orientation of the bone pin 102 by one, two, three or even more degrees of freedom. The degrees of freedom of movement provided to the clamp 100 to match the orientation of the bone pin 102 can accomplish flexibility in the desired trajectories for surgery.

Furthermore, the clamp 100 may include an array throughbore 103 (FIG. 4), through which the array arm 104 may be installed. The array throughbore 103 may include a swage assembly 107, comprising a first swage member 107A and a second swage member 107B, in one example. Thus, swage assembly 107 may be disposed in the array throughbore 103. The first swage member 107A and the second swage member 107B together—as swage assembly 107—may capture or be coupled to the array arm 104.

Moreover, the clamp 100 may include a third opening such as throughbore 105. The throughbore 105 may include threads on an interior thereof. The threads can be complementary to and interact with an example fastener 106. The fastener 106 may be disposed within throughbore 105.

In the example shown, the array arm 104 can have a hexagonal cross-sectional shape. The cross-section is taken perpendicular to a longitudinal central axis of the array arm 104. Other cross-sectional shapes may be used. For example, the array arm 104 may have a round cross-sectional shape and still be capable of being locked in a physical relationship with bone pins 102. Further, the array arm 104 may be composed of a variety of high modulus materials. For example, the array arm 104 may be composed of metals, carbon fiber, reinforced plastic, etc.

Embodiments of the pin channel 101 can have a respective bone pin 102 that can extend substantially perpendicular to the array throughbore 103 containing array arm 104. Further, throughbore 105 within which the fastener 106 can reside can be substantially perpendicular to both pin channel 101 and array throughbore 103.

FIG. 5 shows a cross-sectional view of an embodiment of the clamp 100 from FIG. 4, but with the entirety of bone pin 102 displayed. In the example, bone pin 102 includes distal end 108 including external threads. Distal end 108 of bone pin 102 may be oriented to enter a bone at a desired angle. Additionally, distal end 108 of bone pin 102 may be disposed within the bone.

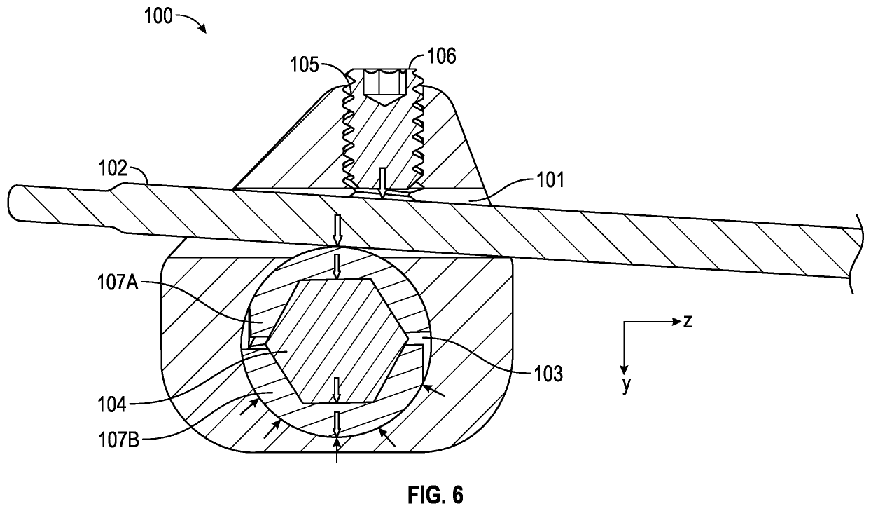

In addition, the clamp 100 may include an overlap or a passageway (FIG. 6) that extends between pin channel 101 and array throughbore 103. A portion of a semi-spherical or hemispherical outer surface of first swage member 107A—may protrude through the passageway into pin channel 101, as shown.

The passageway may include passageway axis (see vertical arrows on left side of FIG. 6). As shown by the single arrow on the right side of FIG. 6, the throughbore 105 can have a counterbore axis. In some examples, the counterbore axis of throughbore 105 and the passageway axis can be offset (e.g., linearly, angularly, etc.) from each other where they intersect the pin channel 101. In other examples, the counterbore axis of throughbore 105 and the passageway axis can be parallel to each other.

FIG. 6 also depicts the interacting forces present in one example of the clamp 100, in accordance with at least some embodiments. In this example, fastener 106 can press on the bone pin 102 via a lower face of the fastener 106. In this example, the fastener 106 can be off-center with respect to the top of the first swage member 107A to force bone pin 102 to seat and lock in a stable manner. The bone pin 102 can transfer the force to the first swage member 107A through contact with the outer surface of the first swage member 107A. The first swage member 107A can press against the array arm 104. The inner surface of the first swage member 107A may be designed to have multiple angled surfaces to provide increased friction against array arm 104. This increased friction may act as additional force to lock array arm 104 into place.

The array arm 104, in turn, can push against the inner surface of the second swage member 107B. The outer surface of the second swage member 107B may then push against the inner surface of the array throughbore 103 and thereby complete the locking chain. Further, the array throughbore 103 may push back against the array arm 104 via reacting, normal forces. Such reacting, normal forces point radially inward relative to the array throughbore 103. As a result of these reacting, normal forces, the array arm 104 may push back against the inner surface of first swage member 107A via a second reacting, normal force. As a result of this second reacting, normal force, the outer surface of the first swage member 107A can push back against bone pin 102. These forces, acting together, lock the bone pin 102 and the array arm 104 securely into place. The separate and distinct swage members 107A and 107B have gaps between them to enable slight relative movement therebetween, thus directing the locking force through the array arm 104.

The swage members 107A and 107B may be designed and constructed such that they may be initially loosely assembled and/or installed in array throughbore 103 for greater range of motion, and then held in place by way of the array arm 104 inserted therethrough until they are ready to be locked in place.

Embodiments of the clamp 100 can enable the bone pin 102 to be oriented in a manner such that an axis of bone pin 102 is not parallel with that of the bone pin channel 101. Specifically, FIG. 6 shows that bone pin 102 may translate in the Y direction to allow bone pin 102 to touch against swage member 107A and move it enough to lock as described above. There is no need for bone pin 102 to move in the X direction, thus through hole 101 may be oval, in some embodiments.

In some embodiments, the longitudinal central axis of the pin channel 101 can be parallel to the z axis (FIG. 6). The central axis of the array throughbore 103 can be parallel to the x axis. The longitudinal central axis of the throughbore 105 can be parallel to the y axis. In some examples, the longitudinal central axis of the pin channel 101 can be perpendicular to the central axis of the array throughbore 103, which can be perpendicular to the longitudinal central axis of the throughbore 105. In some examples, the array throughbore 103 may be larger (along the y axis) than the array arm 104, which enables not only placing of the swage members 107A and 107B in the array throughbore 103 prior to sliding the array arm 104 through the swage assembly 107, but also (once assembled) enables the array arm 104 to have a range of motion (prior to locking the fastener 106) about a rotational axis parallel to the y axis and relative to the clamp 100.

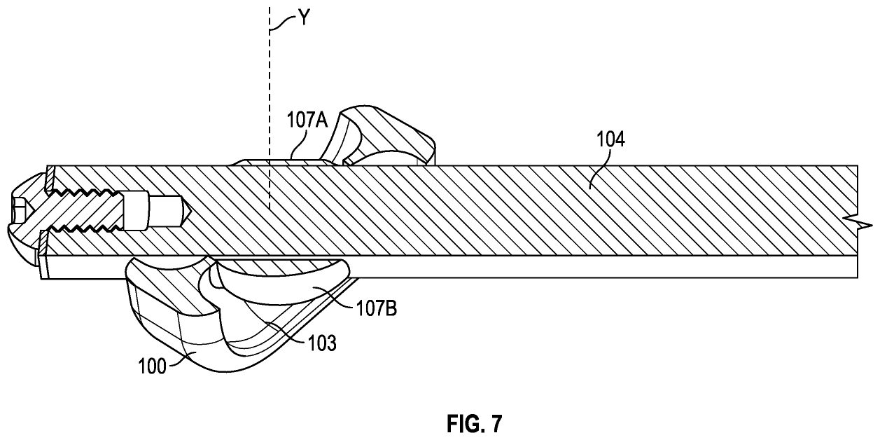

FIG. 7 shows a cross-sectional perspective view of the clamp 100, with the sectional view taken through array arm and looking toward the bottom of the clamp 100. This view includes the lower portion of the clamp 100, the second swage member 107B and the array arm 104. In example systems, an inside surface of the array throughbore 103 is designed and constructed to form a bearing surface that interacts with an outside surface the swage assembly 107. In the example case of the swage assembly 107 defining a spherical outer surface (notwithstanding the gaps between the swage members), an inside surface of the array throughbore 103 defines a bearing surface that is at least partially complimentary in shape to the spherical outer surface of the swage assembly. The example of the clamp 100 can define a bearing surface that enables the swage assembly 107 and array arm 104 to move or rotate within the clamp 100, such as rotation about the y axis.

The system 500 enables the surgeon to determine the location of bone pins 102 without having preset configurations or locations for the bone pins 102 relative to the array arm 104. While in some cases a single femoral array clamp 100 may be used, in other cases two or more clamps 100 can be used. In an instance where more than one clamp 100 is used, a method for using the clamps 100 can include sliding the pin channels 101 of the clamps 100 over the bone pins 102 that are installed within the underlying bone; and, optionally, inserting the array arm 104 through the array throughbores 103 of the clamps 100 (this step may be pre-established or pre-assembled, prior to surgery, in some embodiments); and locking a physical relationship between the bone pins 102 and the array arm 104 by actuating fasteners 106 in the clamps 100.

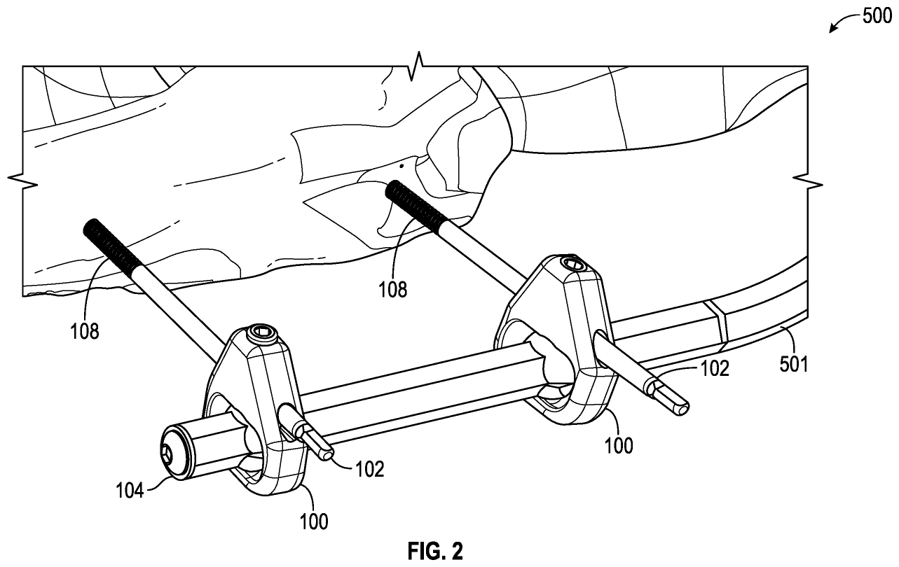

In some examples, bone pins 102 within the system 500 may be tapered (e.g., tapered outwards) from the threaded portion of bone pin 102 threaded portion on distal end 108. This tapering outwards can increase the stiffness of bone pin 102. The distal ends 108 of bone pins 102 may be disposed within the bone (FIG. 2) while the bone pins 102 are locked in a relationship within the clamps 100. The distal ends 108 of the bone pins 102 are disposed within the bone, while the rest of system 500 may be external to the patient.

In these examples, the first swage member 107A may include a feature that can operably mate with a respective bone pin 102 to further lock the components of femoral array clamp 100. In some examples, first swage member 107A and second swage member 107B may each define a semi-spherical or hemispherical outer surface. In such examples, first swage member 107A and second swage member 107B may still be used concurrently within the femoral array clamp 100.

In certain configurations of system 500, the system may further include frictional components within the pin channel 101. The frictional components may add friction to restrict the motion of bone pin 102 while it is disposed within pin channel 101. Examples of frictional components include an O-ring, a ball plunger, a spring loaded device, packing, a mechanical gasket, or combinations thereof. Similarly, frictional components can be included between the array arm 104 and the array throughbore 103, and/or between the array arm 104 and one or both swage members 107A, 107B to reduce any extraneous free play or movement of the femoral array clamp 100 when the user is engaging the bone pins 102.

In certain configurations of system 500, the system may further include a stabilizer to hold fastener 106 in place within counterbore 105. In some examples, counterbore 105 may include threads on an inner diameter. From this, fastener 106 may interlock with the threads upon rotation of fastener 106. In certain examples, fastener 106 may be a set screw.

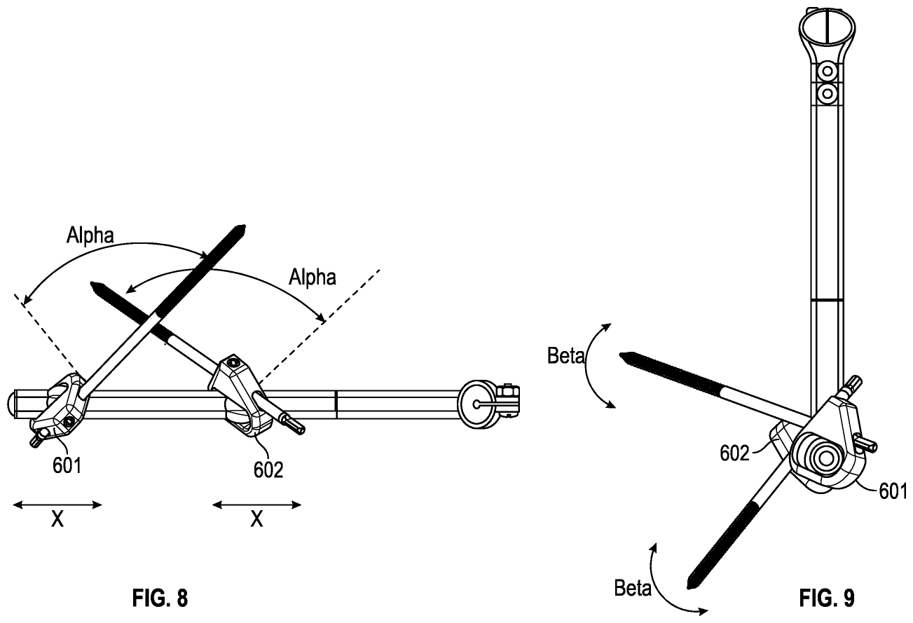

FIGS. 8 and 9 show several ranges of motion for the system. For example, each clamp 601, 602 can have an adjustable angle alpha (generally, horizontally) to alter the orientation of the pin channel. Each pin channel can adjustably swivel to any angle in the alpha range. In addition, the clamps 601, 602 can be independently repositioned linearly along the x-direction, which is generally horizontally. Each clamp 601, 602 also can have another angular range of motion beta (generally, vertically) to alter the orientation of the clamps 601, 602 relative to their respective bone pins, such that their pin channel axes may be adjustable like swivels.

In some examples, the first clamp 601 may be linearly situated at one portion of the array and the second clamp 602 may be situated at another portion of the array.

Since surgeons have wide variations in their practices for placing and situating bone pins, the flexibility of the various degrees of freedom in the system can present a benefit for those in the profession. For example, with the possible different positions illustrated, medical professionals may be cognizant and proactive about concerns such as soft tissue damage, bone fracture, workflow interference, and combinations of the like. This design allows surgeons and other medical professionals to variably install the bone pins in positions along three-dimensional axes based upon their preferred techniques. In some embodiments, a tubular spacer or separator 130 (FIG. 1; shown transparent for illustration purposes) may be positioned over or around the array arm 104 such that clamps 601 and 602 can never be closer together than a predetermined distance. For example, if the clamps were too close or adjacent each other, there could be insufficient rigidity in the system.

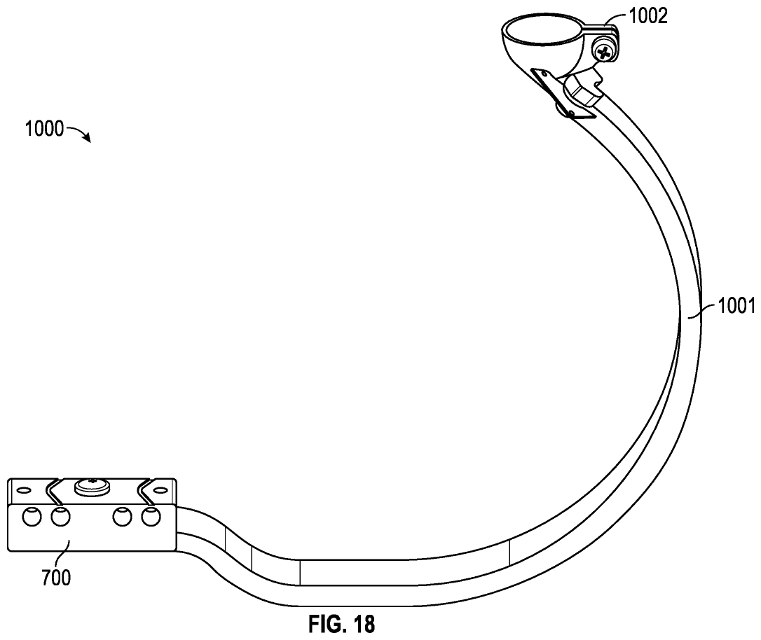

FIGS. 10-17 depict other examples of a femoral bone clamp that can be utilized in a system 1000 (FIG. 18) for femur repair. Like system 500, system 1000 allows the surgeon to position bone pins within the clamps 700 so that the system 1000 and bone pins are locked in a physical relationship that eliminates extraneous motion. With this system 1000, the surgeon can have an increased awareness of the stability and positioning of the distal ends of the bone pins during surgery.

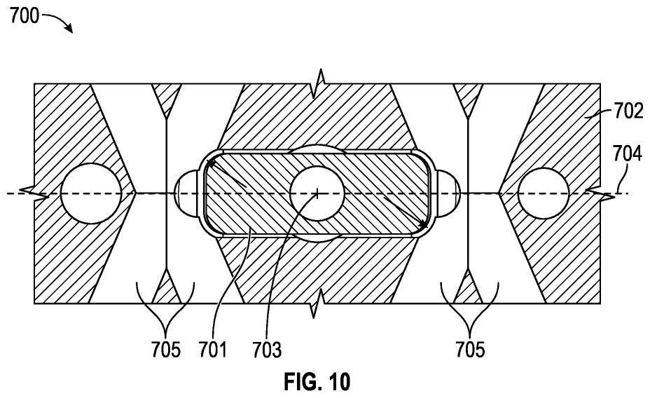

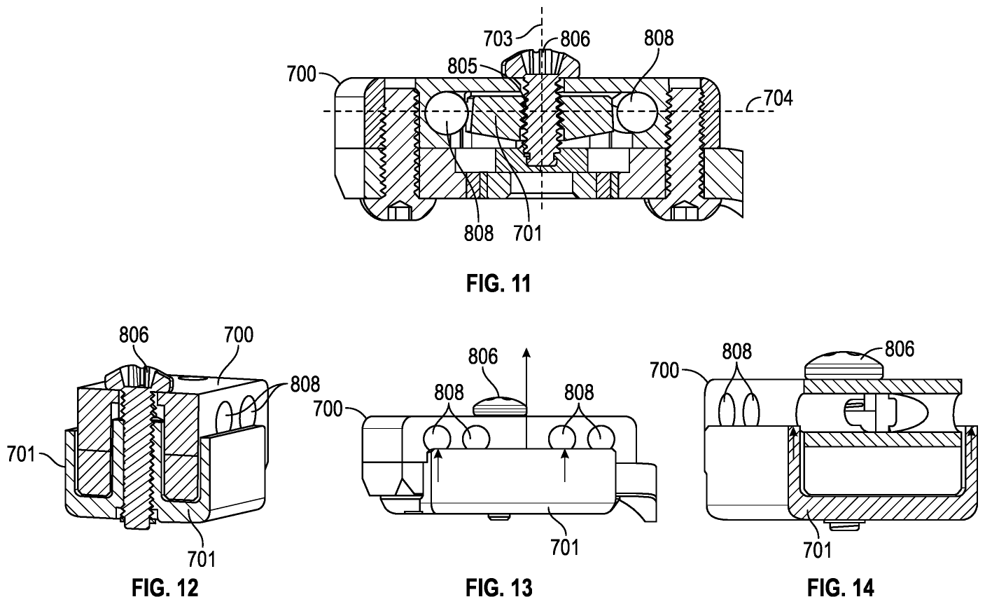

Embodiments of femoral bone clamp 700 can include a wedge 701 in a pin housing 702. In some examples, wedge 701 may move along a wedge axis 703 (FIGS. 10 and 11) and expand along a perpendicular axis 704 to apply force to bone pins in pin housing 702. The force applied by wedge 701 can lock the bone pin in a physical relationship with respect to the clamp 700. In some examples, a femoral array clamp 700 may include a plurality of pairs of bone pin channels 705 that respectively intersect, as shown.

In some examples, the pairs of intersecting pin channels 705 can allow the femoral bone clamp 700 to be fitted with bone pins that are parallel or skewed in various ways. FIG. 10 shows parallel skewed pin channels 705 which may accommodate both left and right hip applications. Versions of the pairs of intersecting pin channels 705 can allow the femoral bone clamp 700 to be fitted with bone pins such that the distal portions of bone pins 108 converge toward each other. Other versions of the pairs of intersecting pin channels 705 can allow the femoral bone clamp 700 to be fitted with bone pins having distal portions 108 that diverge away from each other.

In some examples, moving a fastener 806 (FIG. 11) in wedge 701 can cause the wedge 701 to press against the bone pins so as to secure them within the femoral bone clamp 700.

Wedge 701 can include a counterbore 805 for fastener 806. Fastener 806 can apply force against movable wedge 701 to lock wedge 701 in a physical relationship within femoral array clamp 700. In the locked position, wedge 701 reduces the amount of clearance space through the pin channels 808. In doing so, wedge 701 applies force to the bone pins located in respective ones of the pin channels 808.

FIGS. 12-14 show alternate embodiments of clamp 700 and wedge 701 that function in a manner similar to that described for the embodiment of FIG. 11. However, these versions secure the bone pins 102 with a pinching action rather than a wedging action like FIG. 11. In addition, the embodiment of FIG. 10 can comprise a slightly tapered wedge 701, as indicated by the double lines around wedge 701. The taper enables the wedge 701 to slidingly wedge against the bone pins 102, where the two arrows indicate force at the corners of the wedge 701. In contrast, the embodiment of FIG. 14 can provide three points of contact for each bone pin 102 (e.g., one point of contact on each end of the wedge 701, and one point of contact with pin channel 808).

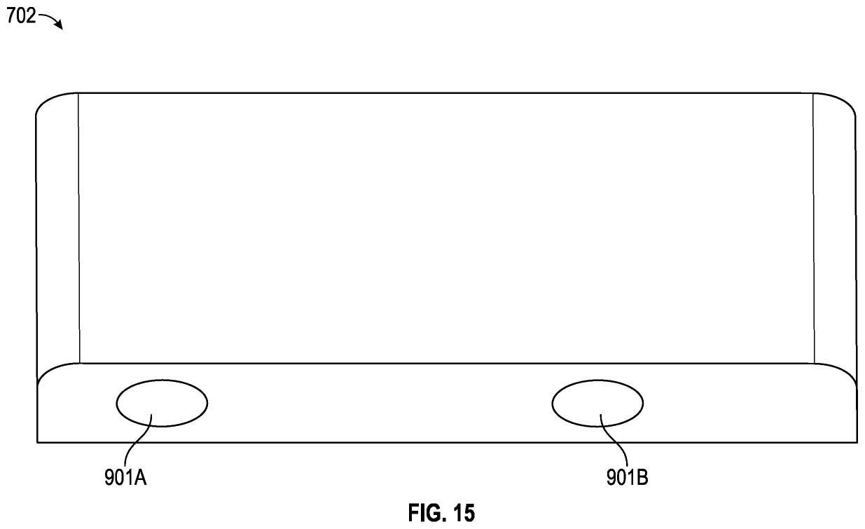

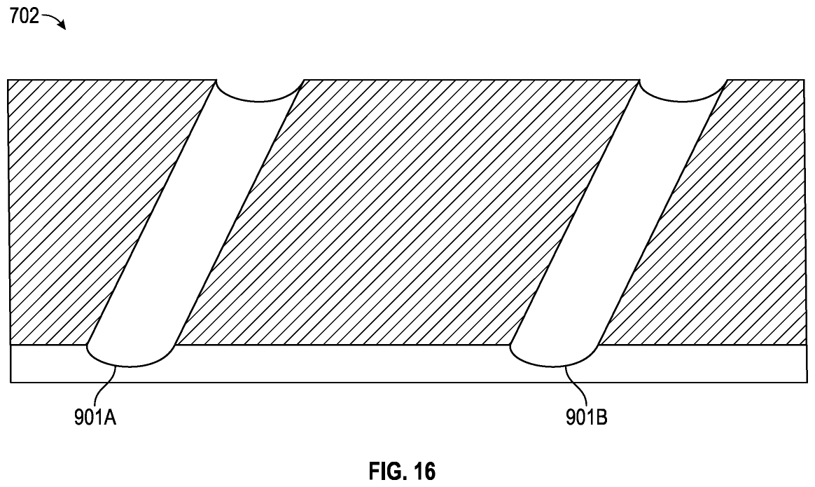

FIGS. 15-17 show other embodiments of pin housings 702 for use in a femoral array clamp. In the example of FIG. 15, pin housing 702 includes parallel pin channels 901A and 901B that can guide respective bone pins. Parallel pin channels 901A and 901B may be throughbores of pin housing 702. The parallel positioning is shown in the cross-sectional view of FIG. 16, in which parallel pin channels 901A and 901B are throughbores.

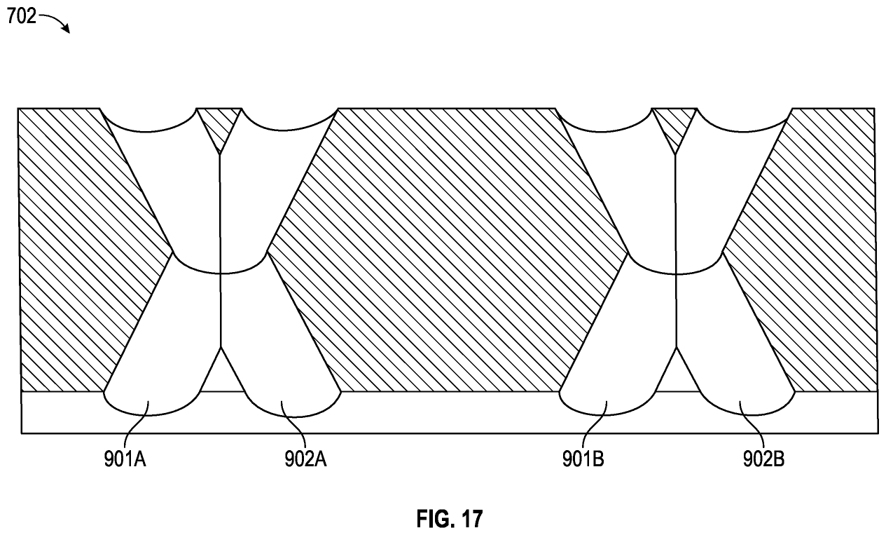

In the embodiment of FIG. 17, the pin housing 702 has a mirrored set of parallel holes, as shown by parallel pin channels 901A and 901B crossing with parallel pin channels 902A and 902B. Pin channels 901A and 902A cross each other, and pin channels 901B and 902B cross each other in pin housing 702. The mirrored pattern of the pin channels 901A, 902A, 901B, 902B may be used to accommodate both left and right hips for surgery. In some examples, only pin channels 901A and 901B, or pin channels 902A and 902B may contain bone pins at one time.

In addition to the clamp 700, system 1000 (FIG. 18) can include C-bracket array 1001 and cup 1002. In certain examples, a tissue protector may be incorporated into base 700 of the clamp. In another example, the tissue protector may include tubing that flips to the left or right sides. The tissue protector can include separate tubes that insert through the pin channels. The tissue protector can be a separate device from clamp 700 that is also included with the system 1000.

In some examples of system 1000, the pin channels can be non-round in shape and/or larger than standard-size bone pins to accommodate various brands, types, and styles of bone pins.

In other examples of system 1000, the bone pins within clamp 700 may be in a locked position that is perpendicular to C-bracket array 1001. In certain examples, the C-arm array 1001 can be non-planar, such as the curved “C” shape that is shown.

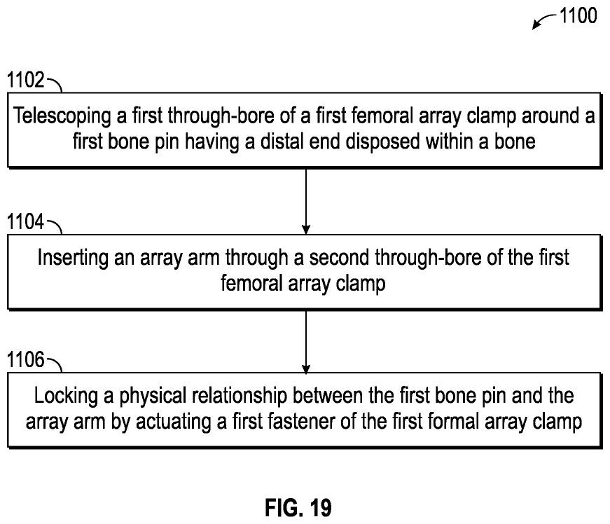

FIG. 19 is a flow chart of a method in accordance with at least some embodiments. Some or all of method 1100 may be performed in preparation for a surgery. In particular, example method 1100 includes sliding a first throughbore of a first femoral array clamp along a first bone pin having a distal end installed in a bone (step 1102). During this step 1102, the femoral array clamp can be coupled to the bone pins embedded in the bone.

Example method 1100 can then include inserting an array arm through a second throughbore of the first femoral array clamp (step 1104). During this step 1104, a portion of an array may be rotated into its vertical position. The example method 1100 also can include locking a physical relationship between the first bone pin and the array arm by actuating a first fastener of the first femoral array clamp (step 1106). In this locking step 1106, the position of the external array can be locked with respect to the femoral array clamp.

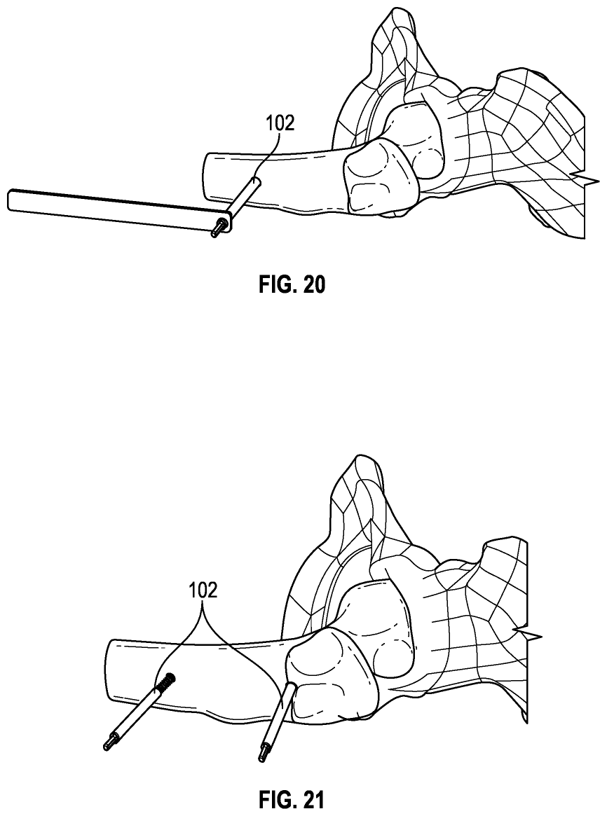

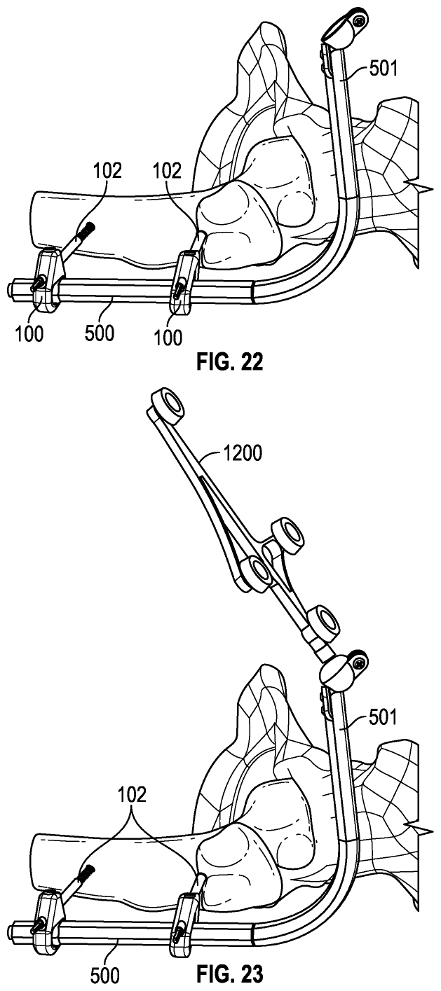

FIGS. 20-23 depict perspective views of sequential steps of a version of a method of using an embodiment of the system 500 in operation. For example, the method may begin as shown in FIGS. 20 and 21 by installing first and second bone pins 102 in the femur. A removable soft tissue protector (e.g., tube) can be used, if desired. As shown in FIG. 22, the system 500 may be located by sliding the clamps 100 onto the installed bone pins 102. The J-bar portion 501 can be rotated into its vertical position without disturbing the clamps 100 or bone pins 102. Next, the clamps 100 are locked onto their positions on the bone pins 102 by tightening the fasteners 106 as described elsewhere herein. As shown in FIG. 23, the tracking array 1200 can then be mounted and locked to the J-bar portion 501 so that the femur surgery may proceed.

The disclosed embodiments have an advantage since the largest bone mass on the femur near the surgical site is the greater trochanter region. Placing bone pins in the greater trochanter allows the bone pins to be introduced with low precision and with minimal fear of iatrogenic damage. The greater trochanter is also close to the skin surface which reduces soft tissue damage, reduces pin deflection, and provides ease of imaging for hip surgery.

Various examples are directed to methods and systems for using a femoral array clamp. In particular, various examples are directed to using a femoral array clamp by sliding a first throughbore of a first femoral array clamp around a first bone pin having a distal end disposed within a bone, inserting an array arm through a second throughbore of the first femoral array clamp, and locking a physical relationship between the first bone pin and the array arm by actuating a first fastener of the first femoral array clamp. The locking of the physical relationship may include compressing a swage assembly. The swage assembly may surround the array arm, and the swage assembly may be disposed within the second throughbore. In other examples, the swage assembly may include a first swage member defining a hemispherical outer surface and an inner surface, and a second swage member defining a hemispherical outer surface and an inner surface. In such an example, the compressing of the swage assembly may further include pressing the bone pin against the hemispherical outer surface of the first swage member, the pressing by the first fastener. The compressing of the swage assembly may further include pressing the hemispherical outer surface of the second swage member against an inside surface of the second throughbore, the pressing by force provided from the first fastener.

Prior to inserting the pins into the bone, it is beneficial to orient the pins with respect to the bone and array to reflect the orientation that the pins will have upon being inserted into the bone. Such oriented inserting of the bone pins and holding the array in place is especially beneficial in computer-assisted procedures.

The above discussion is meant to be illustrative of the principles and various embodiments of the present invention. Numerous variations and modifications will become apparent to those skilled in the art once the above disclosure is fully appreciated. It is intended that the following claims be interpreted to embrace all such variations and modifications.

1. A method for using a femoral array clamp, the method comprising the steps of:
(a) providing a first femoral array clamp with a second throughbore having an array arm slidably mounted in the second throughbore;
(b) sliding a first throughbore of a first femoral array clamp around a first bone pin having a distal end disposed within a bone; and
(c) locking a physical relationship between the first bone pin and the array arm by actuating a first fastener of the first femoral array clamp.

2. The method of claim 1, wherein the step of locking the physical relationship further comprises engaging a swage assembly that is coupled to the array arm, and the swage assembly is disposed in the second throughbore.

3. The method of claim 2, wherein the engaging the swage assembly further comprises clamping the swage assembly between the first bone pin and an inner surface of the second throughbore.

4. (canceled)

5. The method of claim 4, wherein
(a) the swage assembly comprises:
(i) a first swage member having an outer portion and an inner portion; and
(ii) a second swage member having an outer surface and an inner portion; and
the engaging the swage assembly further comprises (b)
(i) engaging the bone pin against the outer portion of the first swage member; and
(ii) engaging the outer portion of the second swage member against an inner surface of the second throughbore.

6. The method of claim 1 wherein, after inserting the array arm and before locking, the method further comprises adjusting an angle between a longitudinal central axis of the array arm and an axis of the first bone pin, wherein the angle is adjustable with at least two degrees of freedom.

7. The method of claim 1, further comprising the steps of:
(a) sliding a first throughbore of a second femoral array clamp around a second bone pin having a distal end disposed within the bone;
(b) inserting the array arm through a second throughbore of the second femoral array clamp; and
(c) locking a physical relationship between the second bone pin and the array arm by actuating a second fastener of the second femoral array clamp.

8. The method of claim 7 further comprising, prior to locking the physical relationships, adjusting a distance between the first femoral array clamp and the second femoral array clamp along the array arm.

9. A femoral array clamp, comprising:
a housing;
a pin channel extends through the housing and has a pin channel axis;
a throughbore extends through the housing;
a passageway couples the pin channel and the throughbore, and the passageway has a passageway axis;
a counterbore extends in the housing and has a counterbore axis, wherein the counterbore intersects the pin channel
a fastener disposed in the counterbore; and
a swage assembly disposed in the throughbore, a portion of the swage assembly protrudes through the passageway into the pin channel.

10. The femoral array clamp of claim 9, further comprising:
a bone pin disposed in the pin channel, wherein
the bone pin defines a bone pin axis,
the femoral array clamp comprises a relaxed configuration in which the housing is capable of translating along the bone pin axis and the swage assembly is capable of rotating within the throughbore; and
the femoral array clamp comprises a locked configuration in which the fastener engages the bone pin against the swage assembly to prevent translation of the housing along the bone pin and prevent rotation of the swage assembly in the throughbore.

11. The femoral array clamp of claim 9, wherein the counterbore axis and the passageway axis are offset from each other where they intersect the pin channel.

12. The femoral array clamp of claim 9, wherein the pin channel axis is perpendicular to the counterbore axis.

13. The femoral array clamp of claim 9, wherein the pin channel axis is perpendicular to the passageway axis.

14. The femoral array clamp of claim 13, wherein the pin channel axis is perpendicular to the counterbore axis.

15. The femoral array clamp of claim 9, wherein the throughbore comprises an inner surface having a semi-spherical shape.

16. The femoral array clamp of claim 9, wherein the counterbore comprises internal threads, the fastener is a set screw that engages the internal threads, and the counterbore axis is parallel to the passageway axis.

17. The femoral array clamp of claim 9, wherein the swage assembly comprises:
a first swage member having a semi-spherical outer surface and an inner surface, and a second swage member having a semi-spherical outer surface and an inner surface.

18. The femoral array clamp of claim 17, wherein:
the semi-spherical outer surface of the first swage member is operatively positioned within the throughbore to receive a force,
the force stabilizes the first swage member,
the force stabilizes the second swage member, and
the second swage member receives a reciprocal force.

19. A system for surgical repair of a femur, the system comprising:
a femoral array clamp comprising
a housing;
a pin channel extends through the housing and comprises a pin channel axis;
a throughbore extends through the housing;
a passageway extends between the pin channel and the throughbore, and the passageway has a passageway axis;
a counterbore in the housing with a counterbore axis, wherein the counterbore intersects the pin channel and the counterbore axis is parallel to the passageway axis;
a fastener disposed in the counterbore; and
a swage assembly in the throughbore, wherein a portion of the swage assembly protrudes through the passageway into the pin channel;
a bone pin configured to be located in the pin channel; and
an array is configured to traverse the throughbore.

20. The system of claim 19, wherein the swage assembly comprises a first swage member with a semi-spherical outer surface and an inner surface, and a second swage member with a semi-spherical outer surface and an inner surface.

21. The femoral array clamp of claim 20, wherein:
the semi-spherical outer surface of the first swage member is operatively positioned in the throughbore to receive a force,
the force stabilizes the first swage member,
the force stabilizes the second swage member, and
the second swage member receives a reciprocal force.

22. (canceled)
